SEQUENCE LISTING (38 sequences)

SEQ ID NO: 1 - DNA, 20 bp, Artificial Sequence
  S100P L
agacagccatgggcatgatt

SEQ ID NO: 2 - DNA, 21 bp, Artificial Sequence
  S100P R
tcatttgagtcctgccttctc

SEQ ID NO: 3 - DNA, 24 bp, Artificial Sequence
  NK4 L
ctagaattcatgtgcttcccgaag

SEQ ID NO: 4 - DNA, 24 bp, Artificial Sequence
  NK4 R
gcgctcgagtcattttgaggattg

SEQ ID NO: 5 - DNA, 20 bp, Artificial Sequence
  CCL20 L
agagtttgctcctggctgct

SEQ ID NO: 6 - DNA, 22 bp, Artificial Sequence
  CCL20 R
tttttactgaggagacgcacaa

SEQ ID NO: 7 - DNA, 20 bp, Artificial Sequence
  CSPG2(NG2) L
tgttcctcccactacccttg

SEQ ID NO: 8 - DNA, 20 bp, Artificial Sequence
  CSPG2(NG2) R
gggcacagtggtaacgagat

SEQ ID NO: 9 - DNA, 20 bp, Artificial Sequence
  PLAU L
gtggccaaaagactctgagg

SEQ ID NO: 10 - DNA, 20 bp, Artificial Sequence
  PLAU R
tcagcgctgtagtccttgtg

SEQ ID NO: 11 - DNA, 20 bp, Artificial Sequence
  MMP12 L
acacctgacatgaaccgtga

SEQ ID NO: 12 - DNA, 20 bp, Artificial Sequence
  MMP12 R
agcagagaggcgaaatgtgt

SEQ ID NO: 13 - DNA, 20 bp, Artificial Sequence
  ESM-1 L
tgtgacagcagtgagtgcaa

SEQ ID NO: 14 - DNA, 20 bp, Artificial Sequence
  ESM-1 R
catgctccgtgagagaaaca

SEQ ID NO: 15 - DNA, 20 bp, Artificial Sequence
  abhd7 L
agatgctcccattcatcgac

SEQ ID NO: 16 - DNA, 20 bp, Artificial Sequence
  abhd7 R
ctgggaaccatggtatttgg

SEQ ID NO: 17 - DNA, 20 bp, Artificial Sequence
  HCAPG L
tattggtgtgccctttgtga

SEQ ID NO: 18 - DNA, 20 bp, Artificial Sequence
  HCAPG R
cagggatattgggattgtgg

SEQ ID NO: 19 - DNA, 18 bp, Artificial Sequence
  CXCL-3 L
agggaattcacctcaaga

SEQ ID NO: 20 - DNA, 18 bp, Artificial Sequence
  CXCL-3 R
ggtgctccccttgttcag

SEQ ID NO: 21 - DNA, 20 bp, Artificial Sequence
  COL5A2 L
gacctcgtggtgacaaaggt

SEQ ID NO: 22 - DNA, 20 bp, Artificial Sequence
  COL5A2 R
agccgcctgatcttcagtaa

SEQ ID NO: 23 - DNA, 20 bp, Artificial Sequence
  MAGEA2 L
ccagcaaccaagaagaggag

SEQ ID NO: 24 - DNA, 20 bp, Artificial Sequence
  MAGEA2 R
gtcttgggcatgacctgatt

SEQ ID NO: 25 - DNA, 20 bp, Artificial Sequence
  GSN L
cagtggtgtggttccaacag

SEQ ID NO: 26 - DNA, 20 bp, Artificial Sequence
  GSN R
gcttgcctttccagacaaag

SEQ ID NO: 27 - DNA, 20 bp, Artificial Sequence
  CDC2 L
ctggggtcagctcgttactc

SEQ ID NO: 28 - DNA, 20 bp, Artificial Sequence
  CDC2 R
ccattttgccagaaattcgt

SEQ ID NO: 29 - DNA, 20 bp, Artificial Sequence
  CST1 L
ccatggcccagtatctgagt

SEQ ID NO: 30 - DNA, 20 bp, Artificial Sequence
  CST1 R
gaaggcacaggtgtccaagt

SEQ ID NO: 31 - DNA, 20 bp, Artificial Sequence
  MELK L
tggctctctcccagtagcat

SEQ ID NO: 32 - DNA, 20 bp, Artificial Sequence
  MELK R
tagcactggcttgtccacag

SEQ ID NO: 33 - DNA, 20 bp, Artificial Sequence
  ATAD2 L
tatggatggattggacagca

SEQ ID NO: 34 - DNA, 20 bp, Artificial Sequence
  ATAD2 R
agatctgtgggtagcgtcgt

SEQ ID NO: 35 - DNA, 20 bp, Artificial Sequence
  FAP L
tgcaagtaaggaagggatgg

SEQ ID NO: 36 - DNA, 20 bp, Artificial Sequence
  FAP R
gctcttgccatcacagttga

SEQ ID NO: 37 - DNA, 20 bp, Artificial Sequence
  MSN L
atcactcagcgcctgttctt

SEQ ID NO: 38 - DNA, 20 bp, Artificial Sequence
  MSN R
cccactggtccttgttgagt

(19) United States
(12) Patent Application Publication
(10) Pub. No.: US 2011/0306513 A1
(43) Pub. Date: Dec. 15, 2011

(54) NOVEL BIOMARKER FOR LIVER CANCER AND APPLICATIONS FOR SAME

(75) Inventors: Eun Young Song, Seoul (KR); Hee Gu Lee, Daejeon (KR); Young II Yeom, Daejeon (KR); Na Young Ji, Gyeonggi-do (KR); Jung II Lee, Daejeon (KR); Min A. Kang, Gumi-si (KR); Young Joo Kim, Daejeon (KR); Yoon Hee Kang, Daejeon (KR); Jae Wha Kim, Daejeon (KR)

(73) Assignee: Korea Research Institute of Bioscience and Biotechnology, Daejeon (KR)

(21) Appl. No.: 13/139,095

(22) PCT Filed: Dec. 3, 2009
(86) PCT No.: PCT/KR09/07202

§ 371 Date: Aug. 22, 2011

(30) Foreign Application Priority Data
Dec. 10, 2008 (KR) 10-2008-0125045
Aug. 14, 2009 (KR) 10-2009-0075199
Aug. 14, 2009 (KR) 10-2009-0075319

(51) Int. Cl. C40B 30/04; C40B 40/10; C12Q 1/68; C07K 16/40; G01N 33/577; C07H 21/00; C07K 16/18; C07K 16/24; C40B 40/06; G01N 33/574

(52) U.S. Cl. 506/9; 506/16; 506/18; 435/6.12; 435/7.92; 435/7.94; 536/24.33; 536/24.31; 530/389.1; 530/389.2

(57) ABSTRACT

The present invention relates to the elucidation of a gene that can act as a novel marker for liver cancer diagnosis and to diagnostic and prognostic measurements of liver cancer using the same. More specifically, it relates to a diagnosis kit that enables diagnostic and prognostic measurement of a liver cancer using a preparation that measures expression levels of at least one gene selected from a group of liver cancer diagnosis markers consisting of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN and/or a method for diagnostic and prognostic measurement of liver cancer using the same. These have been discovered using normal liver tissues and liver cancer tissues collected from the same liver cancer patient of the present invention and represent the markers whose accuracy and reliability have been greatly improved as markers of liver cancer. The markers of the present invention can be used for the accurate diagnosis and prognosis of liver cancer.

### TECHNICAL FIELD

The present invention relates to a biomarker specific to liver cancer and use thereof.

### BACKGROUND ART

In Southeastern countries including South Korea, Japan, Taiwan, and China, the largest population of patients suffers from liver diseases, such as hepatitis, cirrhosis or liver cancer. The death rate by liver cancer is the fourth-highest around the world by 505,000 patients (WTO, 1997). In South Korea, patients with liver cancer occupy third-largest population of cancer patients by 11.5% (Annual Report of Cancer Statistics in Korea in 2002). A liver cancer has been diagnosed so far based on tissue examination or test with a liver cancer marker such as AFP.

Currently, AFP and PIVKA-II are widely known as the biomarkers for diagnosis, prognosis or evaluation of treatment of liver cancer. However, these markers have yet to meet specificity and sensitivity ideals.

AFP (alpha-fetoprotein) in blood has been widely known for its usefulness in the diagnosis of liver cancer. The AFP is useful in the diagnosis of developed liver cancer, and patients with cirrhosis are also generally asked to take periodical AFP tests for early-diagnosis of liver cancer, in consideration of the fact that 3 to 10% of the patients with cirrhosis develop liver cancer every year. However, the AFP test has excessive false positives since AFP concentration rises not only in liver cancer, but also in positive diseases such as alcoholic hepatitis, chronic hepatitis, or cirrhosis. The positive rates of AFP test do not exceed 50-60% (sensitivity is 29.9% and 65.8% in 20 ng/ml and 400 ng/ml, respectively).

Another well-known marker, i.e., PIVKA-II is the desrcarboxyprothrombin (DCP) which is abnormal prothrombin without coagulation. Independently from the serum AFP, PIVKA-II is reported for sensitivity and specificity of 48.2% and 95.9% in the diagnosis of liver cancer. While there are many biological indicators clinically used at present, the use of these biomarkers is considerably limited mostly because the markers do not reflect all the biological characteristics of the liver cancer. Accordingly, for the purpose of prevention and treatment of ever-increasing liver cancer patients, it is imperative to develop a marker which is more specific to liver cancer than AFP or PIVKA-II and capable of effectively diagnosing liver cancer. It is also important to develop a test reagent to diagnose liver cancer at as early as possible.

Tumor markers have been developed from blood, tissue and excretion. However, there are many tumor markers that are detected or observed in an elevated level even in the absence of cancer. Tumor markers are mainly developed for the purpose of preventing or early diagnosing a cancer, or determining a prognosis of a cancer. Several liver cancer marker candidate proteins and genes have recently been reported under the studies of genomics and proteomics. Researchers including Nan-sun KIM have discovered 14 types of genes that vary expression rate in liver cancer tissue compared to normal tissue, and disclosed in their papers and patent applications (South Korean Patent Publication No. 10-2005-0076876 (‘KR2005-0076876’), 2005: International J. of oncology, 29: 315-327 (2006)) the genes for use in liver cancer diagnosis utilizing the differences in gene expression. The paper and KR2005-0076876 demonstrated usefulness of the 14 genes by RT-PCR, and also proposed use of the 14 genes for the purpose of protein measurement using antigen. However, since these genes are mainly targeting tissues, issues such as release into the blood and diagnosis in blood have not been investigated yet. Development of most currently-available tumor markers has been centered on the differences of gene expression of the tissues. However, considering the characteristics of the biomarker, the high gene expression in tissues does not always lead to a diagnosis of urine or serum. Accordingly, for convenience of diagnosis, it is important to develop a tumor marker secreted from blood or urine, and analysis and diagnosis based on the same are also important.

Various papers and patent(s)/patent application(s) have been incorporated herein in their entirety as a reference. The disclosures of the papers and patent(s)/patent application(s) have been incorporated herein in their entirety to explain the level of technology of the pertinent field of the invention and the concept of the invention more clearly.

### DETAILED DESCRIPTION OF THE INVENTION

The inventors have diligently studied to discover a novel biomarker for use in molecular diagnosis of liver cancer and/or liver cancer with speed and accuracy. As a result, the inventors could confirm that the discovered biomarkers can make an early diagnosis and also a prognosis of liver cancer, and completed the invention.

Accordingly, an object of the invention is to provide a diagnostic or prognostic kit for liver cancer.

Another object of the invention is to provide a method for detecting a liver cancer marker to thus provide information necessary for a diagnosis or prognosis of liver cancer.

Yet another object of the invention is to provide a screening method of a substance for prevention or treatment of liver cancer.

The other objects and advantages of the invention will be explained in greater detail below with reference to the detailed description, the claims and the drawings.

According to an aspect of the invention, a diagnostic or prognostic kit for a liver cancer is provided, which comprises at least one nucleotide sequence selected from the group consisting of S100, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN, a sequence complementary to the nucleotide sequence, a fragment of the nucleotide, or an antibody or an aptamer binding specifically to a protein encoded by said nucleotide sequence.

The inventors have diligently studied to discover a novel biomarker for use in molecular diagnosis of liver cancer and/or liver cancer with speed and accuracy. As a result, the inventors could confirm that the discovered biomarkers can make an early diagnosis and also a prognosis of liver cancer, and completed the invention.

The expression “liver cancer diagnostic or prognostic kit” herein refers to a kit containing a composition for diagnosis or prognosis of liver cancer. Accordingly, the expression “liver cancer diagnostic or prognostic kit” may be interchangeably or mixedly used with “composition for diagnosis or prognosis of liver cancer”.

Throughout the specification, the term “diagnosis” encompasses determining susceptibility of an object to a specific disease or disorder, determining whether or not an object currently has a specific disease or disorder, determining a prognosis of an object with a specific disease or disorder (e.g., characterization of pre-metastatic or metastatic cancer, determining stages of cancer, or determining responsiveness of cancer to treatment), or carrying out therametrics (e.g., monitoring status of an object to thus provide information about treatment effect).

The term “liver cancer” herein refers generally to any cancer originated from liver cells. The liver cancer can be mainly categorized into original liver cancer originating in liver, and metastatic liver cancer in which cancer originated from organs other than liver migrates to the liver. Although causes are unknown, most liver cancers involve cirrhosis, and it has been discovered that patients with cirrhosis, chronic active hepatitis B, or carriers of hepatitis B are more prone to liver cancer. The inventors could confirm that with the markers according to the invention, whether or not an object develops liver cancer can be determined with high sensitivity and reliability.

The terms “diagnostic marker”, “marker for diagnostic purpose”, or “diagnosis marker” encompass any substances that can distinguish liver cancer cells from normal cells, such as, for example, organic biological molecules including polypeptide or nucleic acid (e.g., mRNA, etc.), lipid, glycolipid, glycoprotein, saccharides (mono-saccharides, di-saccharides, or oligo-saccharides, etc.), which exhibit increase pattern in cells with liver cancer. In view of the object of the invention, the liver cancer diagnosis marker is one or more genes selected from a group consisting of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN, which exhibit increased expression in liver cancer cells. These markers may use mRNA regarding any one gene, or any one protein coded by said genes, but preferably be a complex marker including two or more of these markers.

S100P (S100 calcium binding protein P) is the member of the S100 family of proteins which is first extracted from placenta. While 5100 genes are collectively located on chromosome 1q21, S100P is the gene located on 4p16. S100P has the GenBank No. NM_{—}005980, and is known to be involved in protein binding and calcium ion binding. Further, S100P is known to be expressed in a limited pattern which includes expression in placenta and epithelial cells of throat. S100P is also called as MIG9. The mRNA sequence of the gene is NM_{—}005980.2 and the protein sequence is NP_{—}005971.1. Connection of S100P to pancreatic and prostate cancer has been reported, but none has reported as of yet regarding possible use of S100P as a marker for the liver cancer.

Natural killer transcript-4 (NK4) is also known as IL-32, and its initial NK4 transcriptome is reported as being expressed only in stimulated T-cells by PHA or IL-2. However, the recent studies reported the expression in epithelial cells in addition to T-cells, by interferon-γ, IL-1β, and TNF α. NK4 is also known as TAIF. It is the gene belonging to cytokine family, and encodes protein that contains tyrosine sulfation site. It is known that the expression of the protein increases after activation of T-cells as the NK cells are activated or cleaved by IL-2. NK4 is located on chromosome 16p13.3 of 16, and has the sequence NC_{—}000016.8.

Chemokine ligand 20 (CCL20) is protein belonging to chemokind family, and similar to Nod2 genes, expressed in the blood mononuclear cells and intestinal epithelial cells, and is known to involve mainly in chemotaxis of immature dendritic cells or memory T cells and B cells (Schutyser E. et al., Cytokine Growth Factor Rev 14:40926 (2003)).

Chondroitin sulfate proteoglycan 2 (CSPG2) is also known as versican. This proteoglycan with molecular weight exceeding 1000 kDa was cloned as the sequence of core protein of fibroblast CSPG2 was revealed. CSPG2 belongs to lectican protein family, and known as CSPG-2, PG-M, etc. CSPG2 is also called as VCAN, WGN, ERVR, PG-M, and easily accessed based on: complete sequence: NC_{—}000005.8, mRNA: NM_{—}004385.2, and protein: NP-004376. CSPG2 gene is located on chromosome 5q14.3, and has been known to have a mechanism to decrease oxidative-damage by strengthening cell-matrix binding. There has been a report suggesting that some domains of the proteins encoded from the CSPG2 gene promotes blood coagulation, but the role of CSPG2 gene as a direct marker for liver cancer is revealed for the first time in the present invention.

Urokinase-type plasminogen activator (PLAU) is the gene encoding serine protease related to decomposition of extracellular matrix, and its linkage to reduced fibrin-binding affinity and Alzheimer's disease has been reported. The protein encoded by this gene plays a role of converting plasminogen into plasmin. PLAU gene is located on chromosome 10q24, and listed as: complete sequence: NC_{—}000010.9, mRNA: NM_{—}002658.2, and protein: NP-002649.1.

Matrix metallopeptidase 12 (MMP12) is considered very important in various sphysiological diseases as this involves in tissue remodeling including embryonic development, bone formation and uterine remodeling during menstrual period. MMP12, also known as macrophage elastase or metalloelastase, was first cloned in mouse by Shapiro et al. [1992, Journal of Biological Chemistry 267:4664], and cloned in human in 1995 by the same scientists. MMP12 is primarily expressed in the activated macropage, and discovered as being secreted from not only the alveolar macrophage of smokers [Shapiro et al., 1993, Journal of Biological chemistry, 268: 23824], but also the foam cells in arthrosclerosis lesions [Matsumoto et al., 1998, Am J Pathol 153:109]. MMP12 involves in breaking extracellular matrix in the normal physiological operation, and the enzymes encoded in this gene decomposes both the soluble and insoluble elastins. The gene is located within chromosome on 11q22.3, and listed as: complete sequence: NC_{—}000011.8, mRNA sequence: NM_{—}002426.3, and protein: NP-002417.2.

Endothelial cell-specific molecule-1 (ESM-1) is a secreted protein which can promote in vitro cell division activity of hepatocyte growth factor/scatter factor (HGF/SF), and is nown as a protein secreted within proteoglycan form of the chondroitin/dermatan sulfate type. ESM-1 is also known as endocan, and the protein coded by this gene is a secreted protein which is mainly expressed in endothelial cells of lung and kidney. The expression of this gene is regulated by cytokines, suggesting that it may play a role in endothelium-dependent pathological disorders. The corresponding gene is located on 5q11.2, and listed as: complete sequence: NC_{—}000005.8, mRNA sequence: NM_{—}007036.3, protein: NP_{—}008967.1 or CCDS3963.1.

Alpha-fetoprotein, or α-fetoprotein (AFP) is a plasma protein produced by the yolk sac and the liver during fetal development that is thought to be the fetal form of serum albumin. The AFP gene and albumin gene are located in parallel on chromosome 4. AFP binds to copper, nickel, fatty acids and billirubin and is found in monomeric, dimeric and trimeric forms. In humans, fetal AFP levels gradually decrease after birth. Normal adult levels are usually achieved by the age of 8 to 12 months. AFP levels are detected low in normal adults, but the function of AFP in adults is unknown. However, in fetuses, APF protein binds estradiol. AFP is measured in pregnant women through the analysis of maternal blood or amniotic fluid, as a screening test for a subset of developmental abnormalities: it is principally increased in open neural tube defects and omphalocoele and decreased in Down syndrome. It can also be used as a biomarker to detect a subset of tumors such as Hepatocellular carcinoma or endodermal sinus tumor in pregnant women, men, and children. Corresponding gene is located on chromosome 4q11-q13, and listed as: complete sequence: NC_{—}000004.10, mRNA: NM_{—}001134.1, and protein: NP_{—}001125.

C-reactive protein (CRP) is a protein found in the blood, the levels of which rise in response to inflammation (i.e. C-reactive protein is an acute-phase protein). CRP is synthesized by the liver and a member of the pentraxin family of proteins. CRP is used mainly as a marker of inflammation, and used for a diagnosis of cardiovascular disease or diabetes. Corresponding gene is located on chromosome 1q21-q23, and listed as: complete sequence: NC_{—}000001.9, mRNA: NM_{—}000567, and protein: NP_{—}000558.

Abhydrolase domain containing 7 (ABHD7) is also known as EPHSRP or FLJ90341, and is the gene listed as: complete sequence: NC_{—}000001.9, mRNA sequence: NM_{—}173567.3, protein sequence: NP_{—}775838.2, CCDS736.1. The gene is located on chromosomal locus 1p22.1 and known for its hydrolase activity. The possibility of using ABHD7 as a marker of liver cancer has not been known.

HCAPG is a gene which is also known as CAPG, CHCG, NCAPG, FLJ12450, or NY-MEL-3, located on chromosome 4p15.33, and listed as: complete sequence: NC_{—}000004.10, mRNA: NM_{—}022346.3, protein: NP_{—}071741.2, CCDS3424.1. Connection to DNMT3B, HSF2, or the like has been reported, but possibility of using HCAPG as a diagnostic marker of liver cancer has not been known.

Chemokine (C—X—C motif) ligand 3 (CXCL3) is also known as GRO3 oncogene (GRO3), or SCYB3. It is known that ERK phosphorylation is inhibited when the expression of this gene decreases. However, not many have been known about CXCL3, and the linkage of CXCL3 to liver cancer marker is addressed herein for the first time. The gene is located on chromosome 4q21, and listed as: genomic complete sequence: NC_{—}000004.10, mRNA: NM_{—}002090.2, and protein: NP_{—}002081.2, CCDS34007.1.

Collagen alpha-2 type V collagen preproprotein (Col5A2) is a human gene that encodes an alpha chain for fibrillar collagens. That is, COL5A2 encodes alpha chain for one of fibrillar collagens. Fibrillar collagen molecules are trimers that can be composed of one or more types of alpha chains. Type V collagen is found in tissues containing type I collagen and appears to regulate the assembly of heterotypic fibers composed of both type I and type V collagen. Mutations in this gene are associated with Ehlers-Danlos syndrome. COL5A2 gene is located on chromosome 2q14-32 and listed as: complete sequence: NC_{—}000002.10, mRNA: NM_{—}000393.2, and protein: NP_{—}000384.2, and known to produce preproprotein.

Cell division cycle 2 (CDC2) gene involves in a cell division, and mutations in this gene indicate risk of Alzheimer's disease. It has been reported that normal cell division is not observed even when CDC2 existing in inner nerve cells of the Alzheimer patients operate. The gene is also known as CDK1, CDC2, CDC28A, and located on chromosome 10q21.1. Complete sequence is NC_{—}000010.9, mRNA is NM_{—}001786.2 or NM_{—}033379.2, and it is well known in the art that protein can be produced in two isoforms of NP_{—}001777.1 and NP_{—}203698.1.

Cystatin-SN (CST1) is a member of cystatin superfamily which encompasses proteins that contain multiple cystatin-like sequences. Some of the members are active cysteine protease inhibitors, while others do not show this inhibitory activity. This gene is located in the cystatin locus and encodes a cysteine proteinase inhibitor found in saliva, tears, urine, and seminal fluid. CST1 gene is located on chromosome 20p11.21 and listed as genomic sequence: NC_{—}000020.9, mRNA: NM_{—}001898.2, and protein: NP_{—}001889.2 as a precursor, and CCDS13160.1 as a protein.

Maternal embryonic leucine zipper kinase (MELK) is known to regulate proliferation of normal stem cells in the nervous system, and also called as HPK38, KIAA0175. MELK is located on chromosome 9p13.2 and listed as NC_{—}000009.10, NM_{—}014791.2, and protein: NP_{—}055606.1, CCDS6606.1. There are a few reports about linkage of MELK with inhibition of pro-apoptosis or to cell proliferation, but not many studies have been conducted so far regarding MELK.

ATAD2 (ATPase family, AAA domain containing 2) is known as ANCCA, or PRO2000, located on chromosome 8q24.13, and listed as genomic: NC_{—}000008.9, mRNA: NM_{—}014109.2 and protein: NP_{—}054826.2, CCDS6363.1. ATAD2 is known as a member of ATPase family, and has well-conserved 220 amino acid residues, in which the residues are known to act as ATP-binding sites. The protein has one or two AAA (ATPases Associated with diverse cellular activities) domains, and helps to cleave or regulate protein complex by carrying out chaperone-like function. Use of ATAD2 as a marker of a specific cancer has not been reported yet, and the use of ATAD2 as a liver cancer marker is disclosed herein for the first time.

Fibroblast activation protein alpha (FAP), also known as FAPA or DPPIV, is a member of serine protease family. Proteins coded by this gene involves in regulating growth of fibroblast and also in binding of epithelial-mesenchymal during development. FAP is listed as: complete sequence: NC_{—}000002.10, mRNA: NM_{—}004460.2, and protein: NP_{—}004451.2, CCDS33311.1, and known to be located on chromosome 2q23.

Moesin (MSN) is a member of the ERM protein family which includes ezrin and radixin. ERM proteins appear to function as cross-linkers between plasma membranes and actin-based cytoskeletons. Moesin is known to play an important role for cell-cell recognition and signaling, but use of moesin as a marker of liver cancer has never been reported. Moesin is located on chromosomal locus Xq11.2-q12 and listed as: complete sequence: NC_{—}000023.9, mRNA: NM_{—}002444.2, and protein: NP_{—}002435.1, CCDS14382.1.

In one preferred embodiment of the invention, changes in expression of the above-mentioned genes or proteins were analyzed with a biological sample taken from liver cancer patients, and as a result, it was confirmed that the above-mentioned genes or proteins had increased expression by two to nine times higher than a normal control group.

The level of gene expression in the biological sample can be confirmed by measuring an amount of mRNA or proteins, and according to the invention, one or more sequences selected from among the above-mentioned 20 genes used as markers may contain base sequences having homology with these sequences or polypeptide coded therefrom.

The term “sequence homology” used herein refers to sequence relationship between nucleic acids, polynucleotides, proteins or polypeptides, and depending on contexts, may be understood in association with the terms that include: (a) reference sequence; (b) comparison window; (c) sequence identity; (d) percentage of sequence identity; and (e) “substantial identity” or “homologous”.

Regarding ‘(a) reference sequence’, this sequence is used as a basis for sequence comparison. A reference sequence may be a subset or the entirety of a specified sequence; for example, as a segment of a full-length cDNA or gene sequence, or the complete cDNA or gene sequence.

As used herein, “(b) comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions or deletions (i.e., gaps) compared to the reference sequence (which does not comprise additions, substitutions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

The sequence alignment for comparison is already well known in the art. The local homology algorithm of Smith and Waterman, (1981) Adv. Appl. Math 2:482, may conduct optimal alignment of sequences for comparison; by the homology alignment algorithm of Needleman and Wunsch, (1970) J. Mol. Biol. 48:443; by the search for similarity method of Pearson and Lipman, (1988) Proc. Natl. Acad. Sci. USA 8:2444; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, and CLUSTAL program well described by Genetics Computer Group (GCG), 7 Science Dr., Madison, Wis., USA; Higgins and Sharp, Gene, 73: 237-244, 1988. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences (Current Protocols in Molecular Biology, Chapter 19, Ausubel et al., Eds., Greene Publishing and Wiley-Interscience, New York (1995)). It is apparent that the above-mentioned programs or new versions of a novel program will be generally used in the future and in combination with the present invention.

As used herein, “(c) sequence identity” or “identity” in the context of two nucleic acid or polypeptide sequences includes reference to the residues in the two sequences, which are the same when aligned for maximum correspondence over a specified comparison window and can be added, deleted and substituted in a typical manner. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule to be harmful. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “(d) percentage of sequence identity” means the value determined by comparing two optimally aligned sequences over a comparison window, wherein the portion of the polynucleotide sequence in the comparison window may comprise additions, substitutions or deletions (i.e., gaps) as compared to the reference sequence (which does not comprise additions, substitutions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “(e)-(i) substantial identity” or “identity” of polynucleotide sequences in various grammatical forms thereof means that a polynucleotide comprises a sequence that has, for example, preferably at least 70%, more preferably at least 80%, more preferably at least 90%, and most preferably at least 99%, compared to a reference sequence using one of the alignment programs described using standard parameters. One of skilled in the art will recognize that these values can be appropriately adjusted to determine corresponding identity of proteins encoded by two nucleotide sequences by taking into account codon degeneracy, amino acid similarity, reading frame positioning and the like.

Substantial identity of amino acid sequences for these purposes preferably means sequence identity of at least 70%, more preferably, 80%, and more preferably 90%, and most preferably at least 99%. Another indication that nucleotide sequences are substantially identical is if two molecules hybridize to each other under stringent conditions. However, those nucleic acids that do not hybridize under stringent conditions are still substantially identical, if these encode substantially the same polypeptide. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “(e)-(ii) substantial identity” and “identity” in the context of a peptide and various grammatical forms thereof indicates that a peptide comprises a sequence with preferably 70% sequence identity to a reference sequence, more preferably 80%, more preferably at least 90% and most preferably at least 95% sequence identity to the reference sequence over a specified comparison window.

According to the present invention, measuring expression levels of mRNA used for diagnosis of liver cancer refers to confirming presence of mRNA of at least one gene selected from a group consisting of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN and measuring expression levels thereof, to thereby measuring mRNA amount. The analysis methods for the above purpose may include RT-PCR, Competitive RT-PCR, Real-time RT-PCR, RNase protection assay (RPA), Northern blotting, or DNA chip, but not limited thereto.

To diagnose liver cancer according to the present invention, measuring expression levels of expressive protein involves confirming presence of the proteins expressed from the genes of the biological sample and expression levels thereof, and preferably, confirming an amount of protein using antibody that specifically binds to the protein of the above-mentioned gene. The analysis methods for this purpose may include Western blot, enzyme linked immunosorbent assay (ELISA), radioimmunoassay (RIA), radioimmunodiffusion, Ouchterlony immunodiffusion, rocket immunoelectrophoresis, immunostaining of tissues, immunoprecipitation assay, complement fixation assay, fluorescence activated cell sorter (FACS), or protein chip, but the invention is not limited thereto.

According to a preferred embodiment of the invention, the kit is a microarray or gene amplification kit.

If a microarray is implemented as a kit of the present invention, a probe is fixed on a solid-phase surface. If a gene amplification kit is implemented as a kit of the present invention, a primer is included.

A probe or primer used for a diagnostic kit according to the present invention has a sequence complementary to nucleotide sequence selected from a group consisting of: S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN.

According to the invention, the “primer” is a nucleic acid sequence having short free 3′ end hydroxyl group and can form base pair with a complement template. It means a short nucleic acid sequence functioning as a starting point for template strand copying. The primer can initiate DNA synthesis under presence of a reagent for polymerization with appropriate buffer solution and temperature, and four different nucleoside triphosphates. The primers according to the present invention are respective marker gene-specific primers, which are sense and antisense nucleic acids with 7 to 50 nucleotide sequences. The primer may have additional properties without altering the basic properties of primer to act as a starting point of DNA synthesis.

The primers according to the present invention may be chemically synthesized using phosphoramidite solid support method, or other generally-known methods. It is also possible to modify the nucleic acid sequence using many means known in the art. Non-limiting examples of such modifications may include methylation, “caps”, substitution of one or more of the naturally occurring nucleotides with an analog, and internucleotide modification such as, for example, those with uncharged linkages (e.g., methyl phosphonates, phosphotriesters, phosphoroamidates, carbamates, etc.) or with charged linkages (e.g., photphorothioates, phosphorodithioates, etc.). Nucleic acid may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators. Furthermore, the nucleic acid sequence herein may also be modified with a label capable of providing a detectable signal, either directly or indirectly. Exemplary labels include radioisotopes, fluorescent molecules, biotin, and the like.

According to a detailed embodiment of the present invention, the kit for diagnostic or prognostic analysis of liver cancer may include a primer pair specific to one or more of genes selected from a group consisting of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN. To be specific, the primer pair may be represented by SEQ. ID. Nos. 1 to 40 of base sequence listing, as listed in the following table.

TABLE 1

| Primer | Sequence |
|---|---|
| S100P | L: AGA CAG CCA TGG GCA TGA TT |
| | R: TCA TTT GAG TCC TGC CTT CTC |
| NK4 | L: CTA GAA TTC ATG TGC TTC CCG AAG |
| | R: GCG CTC GAG TCA TTT TGA GGA TTG |
| CCL20 | L: AGA GTT TGC TCC TGG CTG CT |
| | R: TTT TTA CTG AGG AGA CGC ACA A |
| CSPG2(NG2) | L: TGT TCC TCC CAC TAC CCT TG |
| | R: GGG CAC AGT GGT AAC GAG AT |
| PLAU | L: GTG GCC AAA AGA CTC TGA GG |
| | R: TCA GCG CTG TAG TCC TTG TG |
| MMP12 | L: ACA CCT GAC ATG AAC CGT GA |
| | R: AGC AGA GAG GCG AAA TGT GT |
| ESM-1 | L: TGT GAC AGC AGT GAG TGC AA |
| | R: CAT GCT CCG TGA GAG AAA CA |
| abhd7 | L: AGA TGC TCC CAT TCA TCG AC |
| | R: CTG GGA ACC ATG GTA TTT GG |
| HCAPG | L: TAT TGG TGT GCC CTT TGT GA |
| | R: CAG GGA TAT TGG GAT TGT GG |
| CXCL-3 | L: AGG GAA TTC ACC TCA AGA |
| | R: GGT GCT CCC CTT GTT CAG |
| COLSA2 | L: GAC CTC GTG GTG ACA AAG GT |
| | R: AGC CGC CTG ATC TTC AGT AA |
| MAGEA2 | L: CCA GCA ACC AAG AAG AGG AG |
| | R: GTC TTG GGC ATG ACC TGA TT |
| GSN | L: CAG TGG TGT GGT TCC AAC AG |
| | R: GCT TGC CTT TCC AGA CAA AG |
| CDC2 | L: CTG GGG TCA GCT CGT TAC TC |
| | R: CCA TTT TGC CAG AAA TTC GT |
| CST1 | L: CCA TGG CCC AGT ATC TGA GT |
| | R: GAA GGC ACA GGT GTC CAA GT |
| MELK | L: TGG CTC TCT CCC AGT AGC AT |
| | R: TAG CAC TGG CTT GTC CAC AG |
| ATAD2 | L: TAT GGA TGG ATT GGA CAG CA |
| | R: AGA TCT GTG GGT AGC GTC GT |
| FAP | L: TGC AAG TAA GGA AGG GAT GG |
| | R: GCT CTT GCC ATC ACA GTT GA |
| MSN | L: ATC ACT CAG CGC CTG TTC TT |
| | R: CCC ACT GGT CCT TGT TGA GT |

The term “probe” as used herein includes linear oligomers of natural modified monomers or linkages, including deoxyribonucleotides, ribonucleotides, and the like, capable of specifically binding to a target nucleotide, and naturally occurring or artificially synthesized. In one embodiment, the probe is preferably a single chain and oligodioxyribonucleotide.

In the examples where probe is used, the probe is hybridized with cDNA molecule. In one embodiment, the appropriate conditions for hybridization may be determined by a series of processes based on the optimization processing. Such processing is carried out as a series of processes by those skilled in the art to establish a protocol to use the same in laboratory. For example, conditions including temperature, concentration of components, time of hybridization and washing, components of buffer solution and their pHs, and conditions of ionic strength, etc., depend on various factors including length of the probe, amount of GC, and target nucleotide sequence. The detailed conditions for hybridization can be referred in Joseph Sambrook, at al., Molecular Cloning, A Laboratory Manual, Cold Spring Harbor Laboratory Press, Cold Spring Harbor, N.Y. (2001); and M. L. M, Anderson, Nucleic Acid Hybridization, Springer-Verlag New York N.Y. (199). For example, among the above-mentioned stringent conditions, more stringent condition refers to hybridizing 0.5 M NaHPO₄, 7% SDS (sodium dodecyl sulfate), 1 mm EDT at 65° C., with washing in 0.1×SSC (standard saline citrate)/0.1% SDS at 68° C., Alternatively, the more stringent condition may refer to washing in 6×SSC/0.05% sodium pyrophosphates at 48° C. Less stringent condition may refer to, for example, washing under condition of 0.2×SSC/0.1% SDS at 42° C.

As used herein, the term “antibody” refers to a specific protein molecule as indicates with respect to antigenic site. For the purpose of the invention, the term “antibody” refers to antigens that bind specifically to marker proteins, and encompasses polyclonal antibodies, monoclonal antibodies and recombinant antibodies.

With the novel liver cancer marker proteins found as explained above, it is possible to easily prepare an antibody using technologies known in the art.

As well known in the art, the polyclonal antibody can be produced by injecting the above-mentioned liver cancer marker protein antigen into an animal and collecting blood from the animal, thereby collecting serum containing the antibody. The polyclonal antibody can be produced from animal host species including goats, rabbits, sheep, monkeys, horses, pigs, cattle, dogs, etc.

The monoclonal antibody can be produced using well known hybridoma method (Kohler and Milstein (1976), European Journal of Immunology 6:511-519), or Phage antibody library (Clackson et al., Nature, 352:624-628, 1991; Marks et al., J. Mol. Biol., 222:58, 1-597, 1991). The antibody produced by the above-mentioned method may be isolated and purified using gel electrophoresis, dialysis, salt precipitation, ion exchange chromatography, hydrophilic chromatography, etc.

Further, in one embodiment, the antibody includes not only a complete form having two full-length light-chains and two full-length heavy chains, but also the functional fragments of the antibody molecule. The “functional fragments of the antibody molecule” as used herein refers to the fragments haring at least a function of binding antigens, such as Fab, F(ab′), F(ab′)2 and Fv.

In one embodiment, the antibody may preferably be a micro particle-conjugated antibody. Further, the micro particle may preferably be colored latex or colloidal gold particle. In one embodiment, the antibody may be any antibody that can measure the level of expression of the protein coded by the known mRNA gene with respect to the above-explained 20 markers, but preferably, the kit may be immunoassay kit, and most preferably, the kit may be Luminex assay kit, protein microarray kit or ELISA kit. The Luminex assay kit, protein microarray kit and ELISA kit contain polyclonal antibody and monoclonal antibody with respect to protein coded from one or more genes selected from the above-mentioned 20 gene group, and secondary antibody with respect to the polyclonal antibody and monoclonal antibody to which marker substance is bound.

In one embodiment, example of the kit may include immunochromatography strip kit, Luminex assay kit, protein microarray kit, ELISA kit, or immunological dot kit, but not limited thereto.

The diagnostic kit may additionally include an essential element to perform RT-PCR. The RT-PCR kit includes primer pairs respectively specific to with respect to marker genes. The primer is a nucleotide having a sequence specific to the nucleic acid sequence of each marker gene, which is approximately from 7 bp to 50 bp in length, and more preferably, approximately from 10 to 30 bp in length. The primer may also include a primer specific to the nucleic acid sequence of a control group gene. Additionally, the RT-PCR kit may include test tube or other appropriate container, reaction buffer solution (with varying pH and magnesium concentration), deoxynucleotide (dNTPs), enzyme such as Taq-polymerase and RT-PCR enzyme, DNAse, DNAse inhibitor, DEPC-water, sterile water, etc.

Further, the kit may additionally include essential element necessary for carrying out DNA chip. The DNA chip kit may include a substrate to which gene or cDNA or oligonucleotide corresponding to a fragment thereof is attached, and a reagent, agent, enzyme, etc to prepare fluorescent-labeled probe. The substrate may include a control group gene or cDNA or oligonucleotide corresponding to a fragment thereof.

Further, the kit may additionally include an essential element necessary to carry out ELISA. The ELISA kit may include an antibody specific to a marker protein. The antibody has high specificity and affinity to each marker protein, and almost no cross reactivity to the other proteins, and is a monoclonal, polyclonal or recombinant antibody. Further, the ELISA kit may include an antibody specific to a control group protein. Additionally, the ELISA kit may include a reagent to detect an attached antibody, such as labeled secondary antibody, chromophores, enzyme (e.g., enzyme conjugated with antibody), and other substances that can bind to substrate or antibody thereof.

Further, the kit may additionally include an essential element necessary to carry out protein microarray to analyze complex marker at the same time. The microarray kit may include an antibody specific to the marker protein bound to solid-phase. The antibody has high specificity and affinity to each marker protein and almost not cross reactivity to the other proteins, and is a monoclonal, polyclonal or recombinant antibody. Further, the protein microarray kit may include an antibody specific to the control group protein. Additionally, the protein microarray kit may include a reagent to detect an attached antibody, such as labeled secondary antibody, chromophores, enzyme (fused with antibody), and other substances that can bind to substrate or antibody thereof. The method for analyzing a sample using protein microarray is to isolate protein from the sample, hybridize the isolated protein with a protein chip to thus form an antigen-antibody complex, and therefore, read the result to confirm presence of protein or level of expression thereof and provide information necessary for a diagnosis of liver cancer.

Luminex Assay is a high-throughput quantitative analysis method which can measure maximum 100 types of analytes of a small amount of non-pretreated sample of a patient (10-20 μl), which provides high sensitivity (unit: pg), is capable of quantitative analysis within short time (e.g., 3 to 4 hours), and can replace conventional ELISA or ELISPOT. The Luminex Assay uses multiplex fluorescent microbead which is capable of concurrently analyzing more than 100 biological proteins (makers) in respective wells of 96-well plate and carries out real-time signaling using two types of laser detectors to distinguish and quantify more than 100 polystyrene beads in different color groups. The 100 beads can be constructed in a manner so that the beads are distinguished as follows: on the one side, there are red fluorescent beads divided into more than ten stages, and on the other sides, there are orange fluorescence beads divided into ten stages, with the beads in varying intensity. Accordingly, the beads between the two sides have respectively varying mixing ratios of red and orange to thus construct 100 color-coded bead set. Antibody of protein as a target of analysis is attached to each bead, so it is possible to carry out protein quantification with immuno-antibody interaction using the same. The sample is analyzed using two lasers, in which one is to detect the bead to find out unique bead number thereof and the other is to detect the protein within the sample reacted with the antibody attached to the bead. Accordingly, it is possible to carry out in vivo analysis of 100 proteins concurrently in one well. This analysis provides advantage of detecting even a very small sample which is 15 μl.

The Luminex kit to carry out Luminex assay according to an embodiment may include antibodies which are specific to marker proteins. The antibody has high specificity and affinity to each marker protein and almost no cross-reactivity to the other proteins, and may be monoclonal, polyclonal or recombinant antibodies. Further, the Luminex kit may include antibodies specific to control group proteins. Additionally, the Luminex kit may include a reagent to detect an attached antibody, such as labeled secondary antibody, chromophores, enzyme (linked to antibody), and other substances that can bind to substrate or antibody thereof. The antibody may be micro particle-antibody conjugate, or the micro particle may be colored latex or colloidal gold particle.

In a liver cancer diagnosis or prognosis kit, the liver cancer diagnostic kit containing immunochromatographic strip for diagnosing liver cancer may be a diagnostic kit characterized at having essential elements necessary to carry out rapid test to reveal the result of analysis within 5 minutes. The immunochromatographic strip may desirably include: (a) a sample pad which absorbs a sample; (b) a conjugate pad which is linked to proteins of one or more gene selected from the croup of 20 genes within the sample; (c) a test membrane having a control line and a test line which includes monoclonal antibody with respect to the protein of one or more genes selected from the group of 20 genes; (d) an absorption pad which absorbs remaining sample; and (e) a support. Further, the rapid test kit including immunochromatographic strip may include antibodies specific to marker proteins. The antibodies have high specificity and affinity to each marker protein and almost no cross-reactivity to the other proteins, and may be monoclonal, polyclonal or recombinant antibodies. Further, the rapid test kit may include antibodies specific to control group proteins. Additionally, the rapid test kit may include a reagent to detect an attached antibody, such as nitrocellulose membrane to which specific antibody and secondary antibody are fixed, membrane attached to beads to which antibodies are attached, and other materials necessary for the diagnosis such as absorption pad and sample pad.

Further, in a preferred embodiment, the liver cancer diagnostic kit may be a diagnostic kit characterized of having essential elements necessary to carry out protein microarray to concurrently analyze complex marker. In one embodiment, the protein microarray kit may include antibodies specific to marker proteins attached to solid phase. The antibodies have high specificity and affinity to each marker protein and almost no cross-reactivity to the other proteins, and may be monoclonal, polyclonal or recombinant antibodies. Further, the protein microarray kit may include antibodies specific to control group proteins. Additionally, the protein microarray kit may include a reagent to detach an attached antibody, such as labeled secondary antibody, chromophores, enzyme (linked to antibody), and other substances that can bind to substrate or antibody thereof. The protein microarray in one embodiment may include polyclonal antibody with respect to the proteins attached to a slide, and enzyme-linked secondary antibody with respect to the polyclonal antibody and monoclonal antibody.

In one embodiment, measuring the level of protein expression by the immunological dot array may preferably include: (a) dotting a biological sample on a membrane; (b) reacting an antibody specific to the protein of one or more gene selected from the group of the 20 genes with the membrane on which the sample is dotted; and (c) adding a secondary antibody to which a label is attached on the reacted membrane and staining. The ELISA assay may preferably be sandwich ELISA assay which includes: (a) adsorbing antibody 1 specific to the protein of one or more gene selected from the gene group having base sequence with respect to the 20 markers onto solid phase; (b) forming antigen-antibody complex by contacting antibody 1 adsorbed onto the solid phase with a biological sample of a patient with presumed liver cancer; (c) treating antibody 2 specific to the protein coded by one or more gene selected from the group of genes having base sequences with respect to the 20 markers to which the labels are attached to attach the same to the complex; and (d) measuring concentration of the protein by detecting the labels. The protein microarray assay may preferably include: (a) fixing polyclonal antibody specific to the protein of one or more gene selected from the group of genes including the 20 markers on a chip; (b) forming antigen-antibody complex by contacting the fixed polyclonal antibody 1 with a biological sample of a patient with presumed liver cancer; (c) treating monoclonal antibody specific to the protein coded by one or more gene selected from the group of genes having base sequences with respect to the 20 markers to which the labels are attached to attach the same to the complex; and (d) measuring concentration of the protein by detecting the labels.

By the above-mentioned assays, it is possible to compare the amount of formed antigen-antibody complex in a normal control with the amount of formed antigen-antibody complex in a patient with presumed liver cancer, and determine an increase of meaningful expression in the protein of liver cancer marker gene and thus diagnose presence of liver cancer of the patient.

As used herein, the term ‘antigen-antibody complex’ refers to a linkage of liver cancer marker protein and antibody specific thereto, and the amount of formed antigen-antibody complex can be quantitatively measured through the size of signal of the detection label.

The detection label may be selected from a group consisting of enzyme, fluorescent material, ligand, luminescent material, microparticle, Redox molecule, and radioactive isotope, but not strictly limited thereto. In an example where enzyme is used as the detect on label, usable enzyme may include, but not limited to, β-glucuronidase, β-D-glycosidase, β-D-galactosidase, urease, peroxidase or alkaline phosphatase, acetylcolinesterase, glucose oxydase, hexokinase and GDPase, RNase, glucose oxydase and luciferase, phosphofructokinase, phosphoenolpyruvate carboxylase, aspartate aminotransferase, phosphenolpyruvate decarboxylase, and β-lactamase. The fluorescent material may include, but not limited thereto, fluorecein, isothiocyanate, rhodamine, phycoerythrin, phycocyanin, allophycocyanin, o-phthalaldehyde, and fluorescamin. The ligand tray include biotin derivative, but not limited thereto. The luminescent material may include acridindum ester, luciferin, luciferase, but not limited thereto. The micro particle may include colloidal gold, colored latex, but not limited thereto. The Redox molecule may include ferrocene, ruthenium complex, biologen, quinone, Ti ion, Cs ion, diimide, 1,4-benzoquinone, hydroquinone, K4 W(CN)₈, [Os(bpy)3]2⁺, [RU(bpy)3]2⁺, [MO(CN)8]4⁻, but not limited thereto. The radioactive, isotope may include ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, ¹⁸⁶Re, but not limited thereto.

Measuring the level of protein expression may preferably be carried out using ELISA assay. The ELISA may include various ELISA assays, including, for example, a direct ELISA using labeled antibody that recognizes antigen attached to a solid support, an indirect ELISA using labeled antibody that recognizes couture antibody from the complex of the antibody that recognizes the antigen attached to the solid support, a direct sandwich ELISA using another labeled antibody that recognizes antigen from the antigen-antibody complex attached to the solid support, or an indirect sandwich ELISA which reacts the antigen-antibody complex attached to the solid support with another antibody that recognizes antigen and then uses labeled secondary antibody that recognizes the another antibody. More preferably, a sandwich ELISA assay may be used, in which antibody is attached to solid support and reacted with a sample, followed by attachment of a labeled antibody that recognizes antigen of the antigen-antibody complex for enzymatical staining, or attachment of labeled secondary antibody with respect to the antibody that perceives antigen of the antigen-antibody complex for enzymatical staining. It is possible to determine whether or not the patient develops liver cancer based on the degree of forming complex of liver cancer marker protein and antigen.

Further, western blot may preferably be used, which may use one or more antibody regarding the liver cancer marker. The entire protein is isolated from the sample, and through electrophoresis, the proteins are divided according to sizes thereof. The proteins are then transferred onto nitrocellulose membrane and reacted with the antibody. By checking the amount of generated antigen-antibody complex using the labeled antibodies, it is possible to determine that liver cancer is developed based on the amount of protein generated due to gene expression. Such detection may be carried out by investigating an amount of marker gene expression in control group and an expression level of marker genes in cells that develop liver cancer, mRNA or protein level may be represented by absolute (e.g., μg/ml) or relative difference (e.g., relative magnitude of signal) of the marker proteins.

Further, immunohistochemistry using one or more antibody regarding the liver cancer markers may preferably be performed. Normal liver tissue and tissue with presumed liver cancer are taken and affixed, and paraffin-embedded block is made in a well-known manner. The block is made into segments of 5 μm thickness, attached onto glass slides, and reacted with the one selected from among the above antibodies in a known manner. The non-reacted antibodies are washed. One of the above mentioned detection labels is used, and labeling of the antibodies is read under microscope.

Further, a protein chip may preferably be used, in which one or more antibody regarding the liver cancer marker is arranged on designated locations on a substrate and immobilized in high density. Analyzing a sample using the protein chip may include isolating protein from the sample, hybridizing the isolated protein with the protein chip to form antigen-antibody complex, and reading the result to see presence of protein or expression degree thereof and determine whether or not liver cancer is developed.

In one embodiment, the biological sample refers to tissue, cells, blood, serum, plasma, saliva, cerebrospinal fluid, or urine, and preferably blood or serum, and most preferably, plasma.

In one preferred embodiment of the invention, the kit may additionally include an AFP nucleotide sequence, a sequence complementary to the nucleotide sequence, a fragment of the nucleotide, or an antibody or an aptamer binding specifically to a protein encoded by the nucleotide sequence, and most preferably, may additionally include AFP and CRP nucleotide sequences, sequences complementary to the nucleotide sequences, fragments of the nucleotides, or antibodies or aptamers binding specifically to the proteins encoded by the nucleotide sequences, to thereby make a diagnosis or prognosis of liver cancer. The liver cancer diagnosis can have greatly increased specificity and accuracy if it is performed by additionally including in the kit AFP and CRP nucleotides or antibodies or aptamers binding specifically to the proteins encoded by the nucleotide sequences.

In one preferred embodiment of the invention, the kit may additionally include a CRP nucleotide sequence, a sequence complementary to the nucleotide sequence, a fragment of the nucleotide, or an antibody or an aptamer binding specifically to a protein encoded by the nucleotide sequence, to make a diagnosis or prognosis of liver cancer.

In one preferred embodiment of the invention, (i) ESM-1 and (ii) AFP and/or CRP are used in combination as a marker, in which case diagnosis of liver cancer can have greatly improved accuracy.

According to another aspect of the invention, a method for detecting liver cancer marker is provided, which includes a step of detecting expression of at least one nucleotide sequence selected from the group consisting of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN in a biological sample of a human to provide information necessary for diagnosis or prognosis of liver cancer.

In one preferred embodiment of the invention, the method may use microarray scheme, gene amplification scheme, or antigen-antibody reaction scheme.

In one preferred embodiment of the invention, the method may additionally include a step of detecting expression of AFP nucleotide sequence, and most preferably, additionally include a step of detecting expression of AFP and CRP nucleotide sequence in one or more marker selected from the above-mentioned biomarker group, to thereby detect the liver cancer marker.

In one preferred embodiment of the invention, the method may additionally include a step of detecting expression of CRP nucleotide sequence, to thereby detect the liver cancer marker.

Since both the method for detecting the liver cancer marker and the liver cancer diagnostic kit use the same markers, overlapping procedures or parts will not be explained herein for the sake of brevity.

According to another aspect of the invention, a method for screening substance for preventing or treating liver cancer may be provided, which may include:

(a) contacting a sample for analysis with cells containing at least one nucleotide sequence selected from the group consisting of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN; and

(b) measuring expression level, of the nucleotide sequence, wherein if high expression of the nucleotide sequence is inhibited, the sample is determined to be a substance for preventing or treating liver cancer.

According to the method of the invention, a sample for analysis is brought into contact with the cells that include nucleotide sequence of the marker according to the invention. Preferably, the cells containing the nucleotide sequence of the marker of the invention are human liver cancer cells. Throughout the description of the screening method of the invention, the term “sample” as used herein refers to unknown substance used in screening procedure to inspect whether or not the unknown substance influences the expression level of the marker of the invention. The sample may include, but not limited to, chemical substance, nucleotide, antisense-RNA, siRNA (small interference RNA), and natural extract.

Next, the expression level, of the marker of the invention is measured from the cells treated with the sample. The measurement of the expression level of the marker of the invention may be carried out in the manner explained above, and if the high expression of the nucleotide sequence of the marker of the invention is determined to have been inhibited as a result of measurement, it can be determined that the sample is a substance for preventing or treating liver cancer.

In one preferred embodiment of the invention, the screening method may additionally include a step at contacting a sample for analysis to cells containing AFT nucleotide sequence in the step (a), and measuring the expression level of the AFP nucleotide sequence in the step (b), and most preferably, may additionally include a step of contacting the sample for analysis to the cells containing AFP and CRP nucleotide sequence in the step (a); and measuring the expression level of the AFP and CRP nucleotide sequence in the step (b), to thereby screen substance for preventing or treating liver cancer.

In one preferred embodiment of the invention, the screening method may additionally include a step of contacting the sample for analysis to the cells containing CRP nucleotide sequence in the step (a); and a step of measuring the expression level of the CRP nucleotide sequence in the step (b), to thereby screen the substance for preventing or treating liver cancer.

Since both the method for screening substance for preventing or treating liver cancer and the liver cancer analysis kit use the same marker, overlapping procedures or parts will not be explained herein for the sake of brevity.

To summarize characteristics and advantages of the invention, these include the following:

(i) The invention provides novel molecular markers regarding liver cancer.

(ii) The invention provide markers which are discovered by using normal liver tissue, and liver cancer tissue collected from the same patients with liver cancer, and which have greatly improved accuracy and reliability as the markers for liver cancer.

(iii) The markers of the invention enable accurate diagnosis and prognosis or liver cancer.

### BRIEF DESCRIPTION OF DRAWINGS

FIGS. 1a-1b illustrate RT-PCR results showing levels of expression of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN markers;

FIGS. 2A and 2B illustrate RT-PCR results showing levels of expression of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN markers;

FIGS. 3A and 3B are results confirmed by western blot regarding expression of liver cancer markers in serums of normal subjects and liver cancer patients, in which N denotes serum of a normal subject, m is a marker, CH is chronic hepatitis, LC is liver cirrhosis, and HCC is hepatocellular carcinoma;

FIGS. 4A to 4E illustrate comparison of one marker from among proteins of normal serum and liver cancer serum by immunological dot assay, in which ‘Normal’ denotes serum collected from a normal subject, ‘Dil.fold’ is a magnitude of dilution, ‘HCC’ is hepatocellular carcinoma, and ‘intensity’;

FIG. 5 illustrates a structure of immunochromatography strip according to an invention;

FIGS. 6A and 6B show representation of reaction curves by ELISA assay, in which FIG. 6a represents ELISA reaction curve of ESM-1 and FIG. 6b represents ELISA reaction curve of CRP; and

FIG. 7 shows ROC (receiver-operating characteristic) curve graph comparing sensitivity and specificity of an example where ESM-1 or CRP is mixed with AFP, in which ‘Hepto R’ denotes CRP, ‘Hepto E’ is ESM-1, ‘HCC’ is hepatocellular carcinoma, and ‘L.CIR’ is ‘liver cirrhosis’.

### BEST MODE

The invention will be explained in greater detail below with reference to examples. The examples provided herein are written only for purpose of explaining the invention in details, and accordingly, it should be obvious for those skilled in the art that the scope of the invention is not limited to the examples provided herein.

### EXAMPLES

Throughout the description, the symbol “%” used herein to represent concentration of a specific substance will be expressed as (weight/weight) % for solid/solid, (weight/volume) % for solid/liquid, and (volume/volume) % for liquid/liquid, unless otherwise specified.

### Example 1

### Discovery of Genes with Excessive Expression of Liver Cancer Using DNA Chip

For cDNA microarray experiment, primary hepatocellular carcinoma (HCC) tissues were obtained from forty liver cancer patients at Seoul St. Mary's Hospital and Chonbuk National University Hospital. The normal liver tissues were obtained from patients other than liver cancer patients at Seoul St. Mary's Hospital. The tissue samples were frozen in liquid nitrogen. The total RNA was isolated using RNeasy mini kit (Qiagen, Hilden, Germany). Differences in gene expression were then investigated based on the cancer tissue of the 40 liver cancer patients and the neighboring tissues using cDNA microarrayer.

### Example 2

### Isolation of mRNA from Tissues and Cells

For RT-PCR, normal liver cells and liver cancer cells were collected from twenty liver cancer patients and mRNA was isolated from total forty tissues.

First, upon extracting the tissues with surgical incision, blood was immediately removed in sterilized in PBS, and frozen in liquid nitrogen. After that, the total mRNA was isolated by Guanidinium method and single-step RNA isolation was carried out. The isolated total mRNA was quantified using spectrophotometer, and reserved in −70 C refrigerator for later use.

Total five liver cancer lines were selected (Chang, HepG2, Hep3B, SKHep1, Huh7) and distributed from Korean Cell Line Bank (KCLB) with address at 28, Yeongun-dong, Jongno-gu, Seoul, Republic of Korea.

10% fetal bovine serum (FBS, Hyclon) and penicillin streptomycin (1 mg/ml, Sigma) were respectively added to optimum culture medium, i.e., DMEM (Invitrogen) or RPMI1640 (Invitrogen), cultured for 5 to 6 days, and total RNA was isolated by Guanidinium method and single-step RNA isolation was carried out. The isolated RNA was quantified using the spectrophotometer as explained above, and reserved in −70° C. refrigerator for future use.

### Example 3

### Comparison of Gene Expression Using RT-PCR

RT-PCR was carried out regarding the liver cancer-specific over-expression genes which were selected based on the result of Example 1.

For the purpose of preparing primers, the entire DNA sequences of the respective genes were obtained from core nucleotide (http://www.ncbi.nlm.nih.gov.) of NCBI, and the primer sequences of these genes were designed through Prmer3 program. Using the primers designed as explained above, PCR (polymerase chain reaction) was carried out to thereby investigate the levels of expression of the respective genes. The respective primer sequences are illustrated in Table 1 provided above.

For RT-PCR, cDNA was prepared, from the mRNA extracted from the tissues and cell lines of Example 2 and through reverse transcription (RT) reaction. The cDNA was prepared using cDNA Synthesis kit (AccuAcript High Fidelity 1st Stand cDNA Synthesis Kit, STRATAGENE).

Using the prepared cDNA and primers, RT-PCR (1 cycle: 94° C. 5 min: 2 to 35 cycles: 94° C. 40 sec, 56° C. 40 sec, 72° C. 30 sec; final extension: 72° C. 7 min).

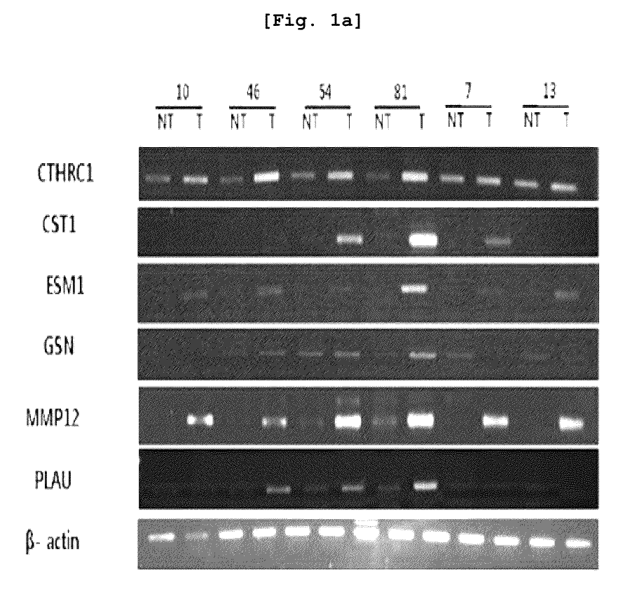

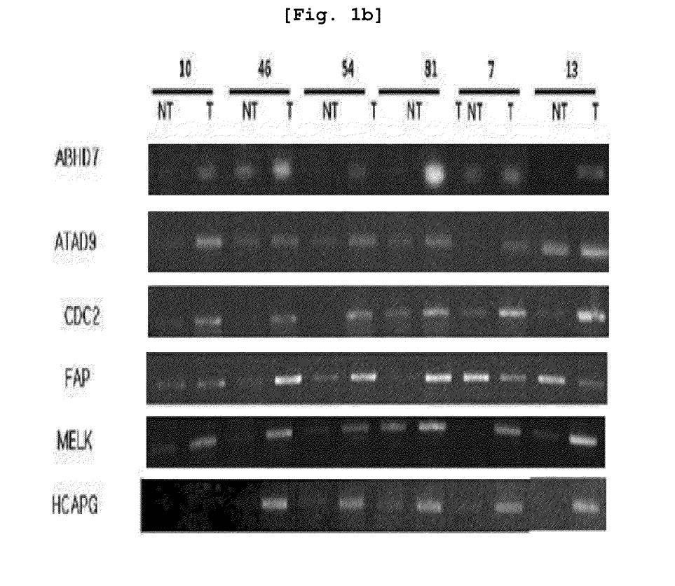

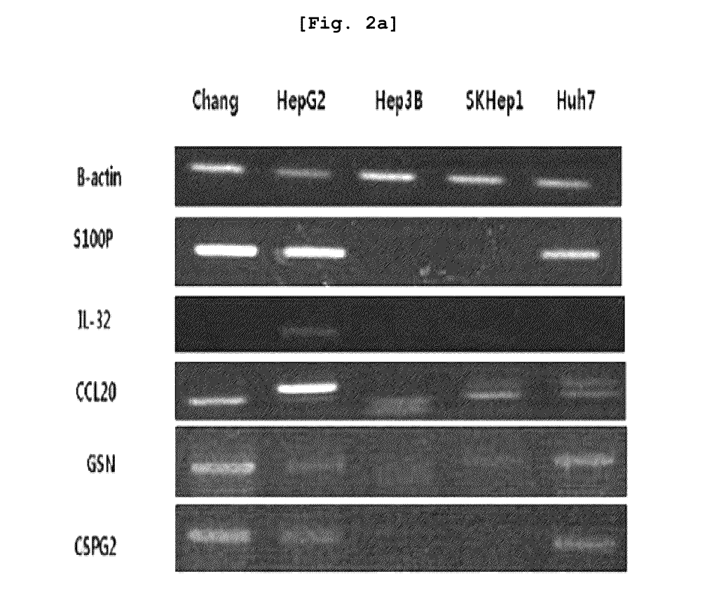

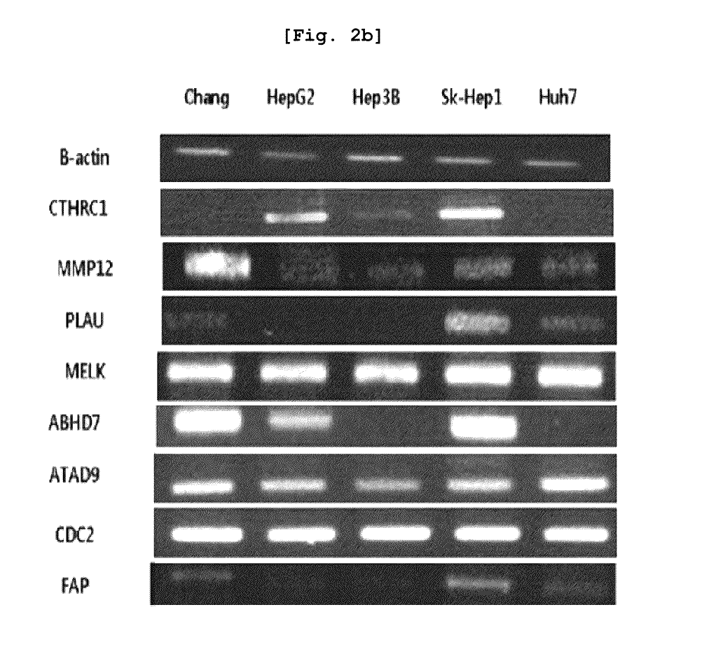

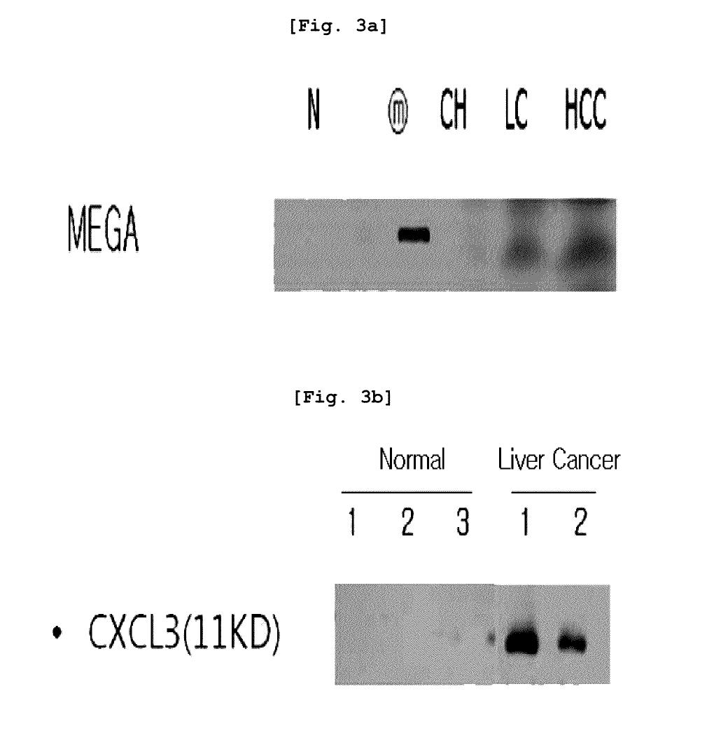

As a result, it was confirmed that there were differences in the gene expressions between normal liver cells and liver cancer cells, and that, in the identical pattern as the result of Example 1, the expression of the twenty genes (i.e., S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN which are anticipated to be the markers) increases in the liver cancer cells than in normal cells (see FIG. 1). The expression of the liver cancer cell line is illustrated in FIG. 2.

### Example 4

### Comparison of Levels of Expression of Proteins within Serum Using Western Blot

The levels of expressions were compared using western blot, among CXCL-3 within serum of liver cancer patient and healthy subjects, and serums of the healthy subjects and patients with chronic hepatitis and patients with cirrhosis.

First, serum was separated from the blood collected from the patients with liver cancer or chronic hepatitis, patients with cirrhosis and healthy subjects, the same volume of sample buffer solution (125 mM Tris pH 6.8, 4$ SDS, 10% glycerol, 0.006% bromophenol blue, 1.8% BME) was added, boiled for 5 minutes, and protein was isolated using 12% electrophoresis (SDS-PAGE). The gel by the electrophoresis which contains proteins isolated based on molecular sizes was contacted with nitrocellulose membrane, and electric current was applied to transfer the proteins onto the nitrocellulose membrane. After blocking for 1 hour in TBST solution (10 mM Tris, 100 mM sodium chloride, 0.05% Tween 20) containing 3% fetal bovine serum (FBS), polyclonal antibody (CXCL 3; Aviva system biology, U.S.A, MAGEA2; Abgent, U.S.A.) regarding antigen was added, and allowed to react overnight while being stirred at 4° C. The remaining antibody was removed by rinsing with PBST, and secondary antibody (Sigma, U.S.A.) with Horseradish peroxydase attached thereto was added and allowed to react for 1 hour at 4° C. while being stirred. After shaking the nitrocellulose membrane with a mixture of ECL solution A (containing Luminol and enhancer) of MILLIPORE and solution B (containing hydrogen peroxide) each mixed in same amount, water is adequately removed from the membrane, and the membrane was attached appropriately onto a film cassette and developed in a dark room. As a result, it was confirmed that while MAGEA and CXCL-3 proteins were not detected in the healthy subject's case, there was over-expression of MAGEA and CXCL-3 in the liver cancer patients. Accordingly, it was obvious that the proteins of the above-mentioned genes can be effectively used as the markers for diagnosing liver cancer.

### Example 5

### Investigating Tissue with Immunostaining

Immunostaining was carried out with respect to the proteins in the normal liver tissue and liver cancer tissue.

First, by surgical incision, normal liver cells and liver cancer cells were extracted from the liver cancer patients and paraffin-embedded block was made. The block was cut into 5 μm thickness using microtome, and attached onto glass slides. That is, tissue slides were prepared. The tissue slides were subject to known immunostaining (in which the slides were de-paraffinized and antigen was detected for 10 minutes on citrate buffer (pH 6.0). Then in order to remove endogenous tissue peroxidase activity, the slides were treated with 3% hydrogen peroxide in methanol, and incubated with 1% BSA to block non-specific binding. The slides were reacted with antibody overnight in 4° C. humid chamber. The slides were stained with standard EnVision-HRP kit (Dako, Glostrup, Denmark) and colored with diaminobenzidine. The slides were contrast stained with 10% Mayer's hematoxylin. The slides were then stained with the same isotype mouse (IgG or antibody diluted solution was used as a negative control group) and presence and location of the proteins within the tissue were inspected under microscope.

### Example 6

### Measurement of Protein in Serum of Patients by Immunodot

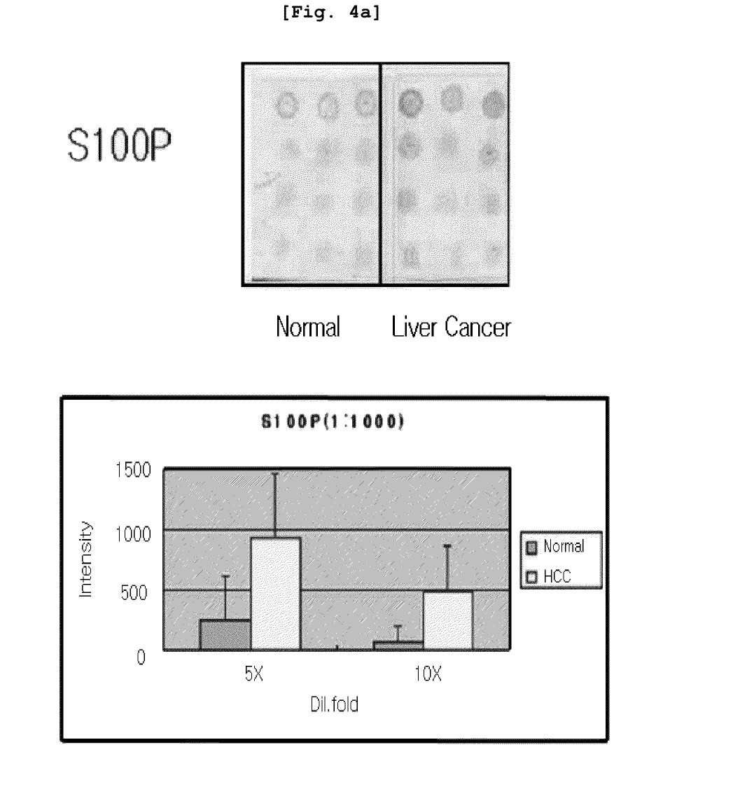

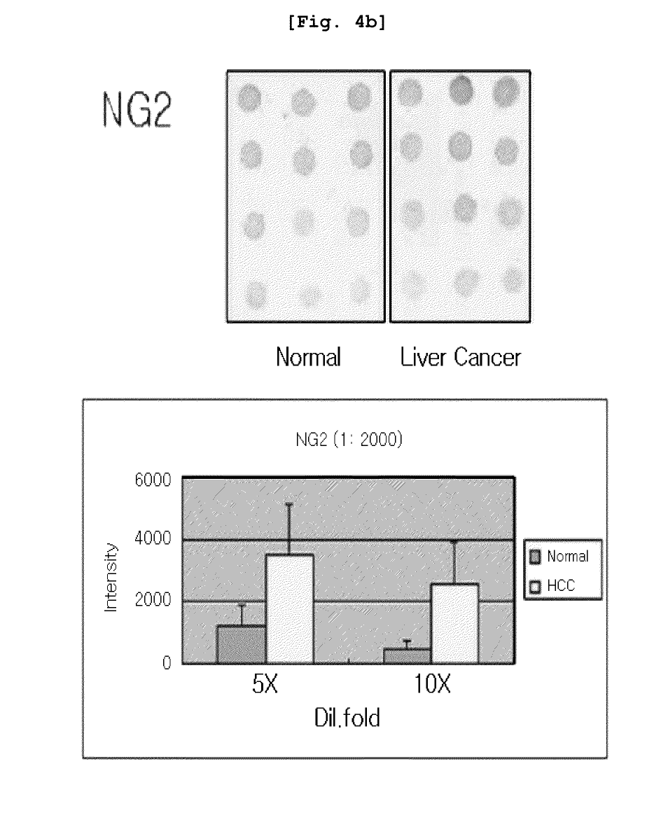

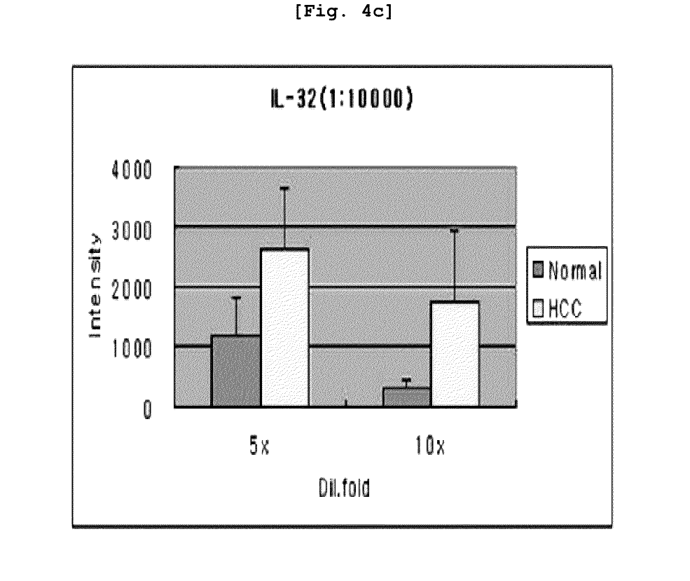

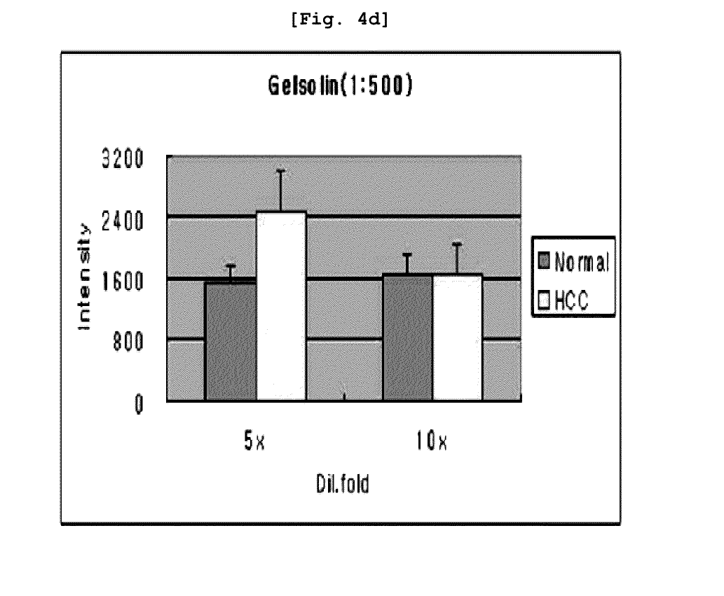

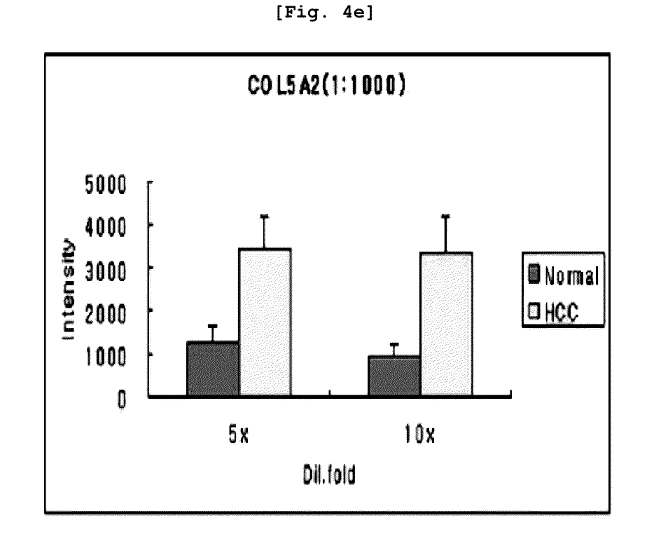

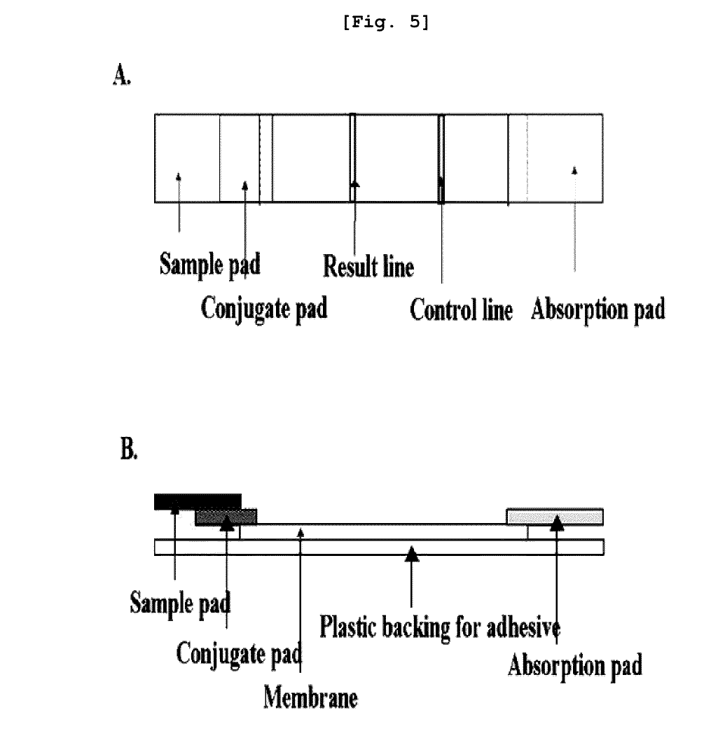

Immunodot method was established using polyclonal antibody, to thereby compare level of secretion of S100P, NK4 (IL-32), CSPG2 (NG2), Col5A2, Gelsolin (GSN)), MSN (Meosin) in normal serum and liver cancer serum. Serum sample (10 pieces per patient) diluted by 5 to 20-fold was dotted each by 2 μl onto nitrocellulose membrane, dried at room temperature, and blocked with 1% BSAT (bovine serum albumin in Tris-buffered saline) solution, Monoclonal antibody (BD, 1:1000) regarding S100P protein, polyclonal antibody (1:10000) regarding NK4 (IL-32) protein, monoclonal antibody (R&D, 1:2000) regarding CSPG2 (NG2) protein, polyclonal antibody (Abcam, 1:1000) regarding Col5A2 protein, monoclonal antibody (Abnova, 1:500) regarding Elsolin (GSN) protein, and monoclonal antibody (Abnova, 1:1000) regarding MSN (Meosin) protein were allowed to react, secondary antibody conjugating horseradish peroxidase (1:10000) was added, and colored with DAB solution (0.5 mq/ml diaminobenzidine in PBT) (Sigma. U.S.A). After scanning, the degrees of coloring were compared (FIG. 4). Referring to FIG. 4, the biomarkers according to the invention had over expression of proteins in liver cancer's cases than in normal cases. Therefore, it was confirmed that the genes or proteins can be used as effective markers for determining a diagnosis or prognosis of liver cancer, Immunodot was carried out regarding CCL20, PLAU, MMP12, ESM-1, abhd7, HCAPG, CXCL-3, MAGEA, MSN (Meosin), CDC2, CST1, MELK, ATAD2 and FAP in the same manner as explained above, and the same result was obtained.

### Example 7

### Establishing ELISA System and Diagnosis of Liver Cancer Using the Same

(7-1) Establishing ELISA System

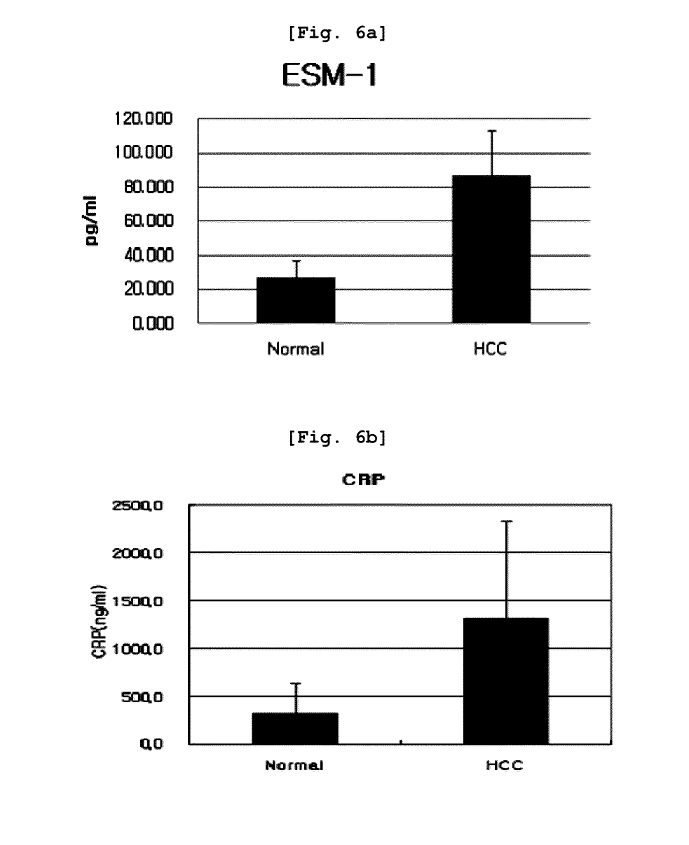

Polyclonal antibodies (1 μg/ml, NK4; Konkuk University, CSPG2; Chemicon, PLAU; Abgent, MMP12, ESM-1; Genetex, ABHD7, HCAPG; Abnova, CXCL-3; Genetex, Col5A2; Genetex, MAGEA: Abnova) regarding NK4, CSPG2, PLAU, MMP12, ESM-1, ABHD7, HCAPG, CXCL-3, Col5A2, MAGEA, GSN, CDC2, CST1, MELK, ATAD2, FAP and MSN were diluted with 0.1 M carbonate buffer (pH 9.6) at concentration of 1 μg/ml, and distributed into 96-well microtiter plate by 100 μl each. After coating at 4° C. overnight, the plate was washed three times with PBS solution (PBS-T) containing 0.5% Tween 20. After blocking at room temperature for two hours with 1% BSA solution, the plate was washed three times using PBS-T solution. The NK4, CSPG2, PLAU, MMP12, ESM-1, ABHD7, HCAPG, CXCL-3, Col5A2, MAGEA, GSN, CDC2, CST1, MELK, ATAD2, FAP and MSN proteins were diluted and added by 100 μl, allowed to react at room temperature for two hours, and washed three times using PBS-T solution. Monoclonal antibodies (1:2000, NK4: Konkuk University, CSPG2; R&D, PLAU; Genetex, MMP12; ESM-1; R&D, ABHD7, HCAPG, CXCL-3; aviva, Col5A2; Genetex, MAGEA: Abnova) regarding NK4, CSPG2, PLAU, MMP12, ESM-1, ABHD7, HCAPG, CXCL-3, Col5A2, MAGEA, GSN, CDC2, CST1, MELK, ATAD2, FAP and MSN proteins were diluted by 100 μl each, allowed to react for two hours and washed. 100 l of secondary antibody to which 2000-fold diluted horseradish peroxidase is attached was added, allowed to react at room temperature for one hour, washed three times, and lastly, colored using TMB solution. The absorbence of the colored samples was measured at 450 nm wavelength using ELISA reader (Molecular device, Sunnyvale, Calif., U.S.A.) (see FIG. 6). Meanwhile, ELISA was carried out regarding S100P (Abnova), CCL20 (R&D Systems), CRP (Immunology consultants laboratory, Inc.) and AFP (Calbiotech, Inc.) using commercially-available ELISA kit.

(7-2) Measurement of Proteins in Patients' Serum Using ELISA System

Concentration of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, CRP, Hyaluronan and AFP proteins in the patients' serum was measured using the ELISA system established in Example 7-1. The serums of healthy subjects and liver cancer patients were diluted 5-fold, and the concentration of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins was calculated.

FIGS. 6a to 6c show the representative examples of ELISA assay results, in which both ESM-1 and CRP proteins are present in the liver cancer patients' serum in high levels, and present in the liver cancer serums in levels higher than the normal serum by approximately 2 to 6 times. The similar high expression was observed regarding the other biomarkers.

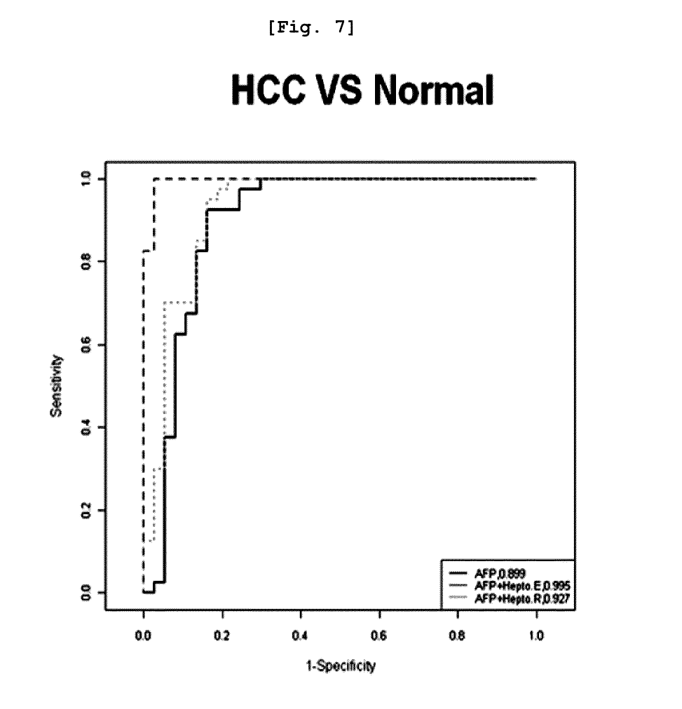

Meanwhile, in order to evaluate the diagnose accuracy of the markers regarding ESM-1, CRP and AFP complex markers, ROC (receiver-operating characteristic) curves were plotted (Stuart G Baker et al., BMC Medical Research Methodology 2:4 (2002); Buyse M et al., J Nat'l Cancer Inst. 98: 1183 (2006)). Referring to FIGS. 7a and 7b, diagnose accuracy increases regarding liver cancer when complex marker is used.

### Example 8

### Preparation of Kit & Measurement of Protein in Serum

(8-1) Sandwich Type ELISA Kit

Kit to measure concentration of NK4, CSPG2, PLAU, MMP12, ESM-1, ABHD7, HCAPG, CXCL-3, Col5A2, MAGEA, GSN, CDC2, CST1, MELK, ATAD2, FAP and MSN proteins was prepared using the following components:

A. Solid-phase antibody: This is a microtiter plate to which antibody is adsorbed. That is, 100 μl polyclonal antibodies (NK4; Konkuk University, CSPG2; Chemicon, PLAU; Abgent, MMP12, ESM-1; Genetex, ABHD7, HCAPG; Abnova, CXCL-3; Genetex, Col5A2; Genetex) regarding NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN proteins were added onto the microtiter plate, left at 4° C. overnight, and albumin was adsorbed onto the space on the solid-phase surface.

B. Detection antibody: Monoclonal antibodies (NK4: Konkuk University, CSPG2; R&D, PLAU; Genetex, MMP12; ESM-1; R&D, ABHD7, HCAPG, CXCL-3; aviva, Col5A2; Genetex, MAGEA: Abnova) regarding NK4, CSPG2, PLAU, MMP12, ESM-1, ABHD7, HCAPG, CXCL-3, Col5A2, MAGEA, GSN, CDC2, CST1, MELK, ATAD2, FAP and MSN proteins.

C. Enzyme conjugated antibody: Secondary antibody solution to which horseradish peroxidase (HRP) is linked (Sigma. U.S.A)

D. Serum dilution solution

E. Substrate solution (TMB)

F. Washing solution: PBS-T containing 0.05% Tween

G. Standard solution: Standard solutions of NK4, CSPG2, PLAU, MMP12, ESM-1, ABHD7, HCAPG, CXCL-3, Col5A2, MAGEA, GSN, CDC2, CST1, MELK, ATAD2, FAP and MSN proteins.

Meanwhile, ELISA regarding S100P (Abnova), CCL20 (R&D Systems), AFP (Calbiotech, Inc.) and CRP (Immunology consultants laboratory, Inc.) was carried out using commercially-available ELISA kit.

The serum dilution reaction of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP in the liver patients was tested with the above-mentioned kit.

Each serum sample was diluted appropriately using serum dilution solution D (containing 1% BSA) on the solid-phase antibody A, added by 100 μl per well, and the concentration of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins was investigated using the sandwich type ELISA kit prepared as explained above in Example 8-1.

(8-2) Immunochromatography Kit

8-2-1. Preparation of Immunochromatography Strip

1) Preparation of Ab-Gold Conjugate

The antibody was added by 15 μg/ml into colloidal gold particles (BBinternationa, UK), allowed to react at room temperature for 2 hours while rotating, followed by addition of 10% BSA (bovine serum albumin) by a volume of 1/10 until 1% concentration was reached, and additional reaction for 1 hour. After centrifuging at 12000 rpm for 40 minutes, the upper aqueous phase was removed, followed by addition of 2 mM borate buffer to wash the Ab-gold conjugate solution. The washing was carried out three times. After the last washing, 2 mM borate buffer containing 1% BSA was added by a volume approximately of 1/10 of the gold solution to give suspension. Absorbence at 530 nm was measured with UV spectrophotometer (Molecular Device, Sunyvale, Calif., U.S.A), and dilution was carried out until absorbence reaches 3.00 for use.

2) Sample Pad

The sample pad is used to absorb the tested sample, and is made from cellulose material. The sample pad may be replaced with any material provided that the material can absorb the sample.

3) Glass Fiber (GF) Membrane

The membrane was treated with 20 mM borate buffer containing sucrose.

4) Nitrocellulose (NC) Membrane and Line Treatment

The nitrocellulose membrane (Millipore) was cut into appropriate size (0.7 cm×5 cm), secondary antibody (Sigma, U.S.A) was line treated at a spot approximately 3.4 cm from a lower end of the plastic backing as a control line, and monoclonal antibodies (S100P, CCL-20, NK4: Konkuk University, CSPG2; R&D, PLAU; Genetex, MMP12; ESM-1; R&D, ABHD7, HCAPG, CXCL-3; aviva, Col5A2; Genetex, MAGEA: Abnova) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins were line treated, and dried so that nitrocellulose membrane was prepared.

5) Absorbent Pad

Cellulose membrane was used to absorb non-reacted substances in the sample after immunological reaction, so that the sample solution including the analyzed substance can be transferred by capillary phenomenon.

6) Plastic Backing for Adhesion

On the adhesion plastic backing of the immunochromatography strip prepared in Example 8-2-1, the sample pad, GF membrane, NC membrane and absorbent pad were stacked in turn, but assembled in such a manner that the substance can be transferred continuously by the capillary phenomenon.

8-2-2. Evaluation of the Result

60-70 μl sample (serum:elution buffer=1:5) was added on the sample pad, and presence of coloring and color intensity were observed after 3 to 5 minutes on the control line and the result line. For the positive sample, red color lines were observed on both the control and result lines, while the red color line was observed only on the control line in the case of negative sample.

(8-3) Luminex Kit

8-3-1. Preparation of Luminex Kit

Polyclonal antibodies (S100P, NK4; Konkuk University, CCL20, CSPG2; Chemicon, PLAU; Abgent, MMP12, ESM-1; Genetex, ABHD7, HCAPG; Abnova, CXCL-3; Genetex, Col5A2; Genetex, MAGEA: Abnova) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN proteins were attached to beads. The sample was diluted and added by 100 μl, allowed to react at room temperature for two hours, and washed three times using PBS-T solution. Monoclonal antibodies (S100P, CCL-20, NK4; Konkuk University, CSPG2; R&D, PLAU; Genetex, MMP12, ESM-1; R&D, ABHD7, HCAPG; CXCL-3; aviva, Col5A2; Genetex, MAGEA: Abnova) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, CRP and AFP proteins were diluted by 100 μl each, allowed to react for two hours, and washed. 100 μl secondary antibody (Molecular probe, U.S.A) to which phycoerythrin (PE) with 2000-fold dilution was attached was added, allowed to react at room temperature for one hour, washed three times, and measured with Luminex device. The relationship between intensity and concentration was expressed in graphical form, and standard curve was calculated.

8-3-2. Sandwich Type Luminex Kit

Kit to measure concentration of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN proteins was prepared using the following components:

A. Solid-phase antibody: Polyclonal antibodies (NK4; Konkuk University, CSPG2; Chemicon, PLAU; Abgent, MMP12, ESM-1; Genetex, ABHD7, HCAPG; Abnova, CXCL-3; Genetex, Col5A2; Genetex, MAGEA: Abnova) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN proteins to which fluorescent beads are attached.

B. Detection antibody: Monoclonal antibodies (NK4: Konkuk University, CSPG2; R&D, PLAU; Genetex, MMP12; ESM-1; R&D, ABHD7, HCAPG, CXCL-3; aviva, Col5A2; Genetex, MAGEA: Abnova) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN proteins.

C. Enzyme conjugated antibody: Secondary antibody solution to which PE is linked (Molecular probe, U.S.A.)

D. Serum dilution solution

E. Substrate solution (TMB)

F. Washing solution: PBS-T containing 0.05% Tween

G. Standard solution: Standard solutions of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins.

(8-4) Protein Microarray Kit

8-4-1. Protein Microarray System

Polyclonal antibodies (NK4; Konkuk University, CSPG2; Chemicon, PLAU; Abgent, MMP12, ESM-1; Genetex, ABHD7, HCAPG; Abnova, CXCL-3; Genetex, Col5A2; Genetex, MAGEA: Abnova) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins were coated on proteagen well chip. After blocking with BSA solution, the serum sample was diluted and added, allowed to react at room temperature for one hour, and washed three times using PBS-T solution. Monoclonal antibodies (S100P, CCL-20, NK4; Konkuk University, CSPG2; R&D, PLAU; Genetex, MMP12, ESM-1; R&D, ABHD7, HCAPG, CXCL-3; aviva, Col5A2; Genetex, MAGEA: Abnova) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins were diluted, allowed to react at 37° C. for one hour and washed. 100 μl of secondary antibody to which 2000-fold diluted Cy3 was attached, was added, allowed to react at room temperature for 0.5 hour, washed three times, and fluorescence at 532 nm was measured. The relationship between intensity and concentration was plotted into a graphical form, from which standard curve was calculated. Using the protein microarray system prepared as explained above, concentration of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins of patients' serum was measured.

8-4-2. Sandwich Type Protein Microarray Kit

Kit to measure concentration of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins was prepared using the following components:

A. Solid-phase antibody: Polyclonal antibodies (S100P, NK4; Konkuk University, CCL20, CSPG2; Chemicon, PLAU; Abgent, MMP12, ESM-1; Genetex, ABHD7, HCAPG; Abnova, CXCL-3; Genetex, Col5A2; Genetex) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins.

B. Detection antibody: Monoclonal antibodies (S100P, CCL-20, NK4: Konkuk University, CSPG2; R&D, PLAU; Genetex, MMP12; ESM-1; R&D, ABHD7, HCAPG, CXCL-3; aviva, Col5A2; Genetex, MAGEA: Abnova) regarding S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins.

C. Enzyme conjugated antibody: Secondary antibody solution to which Cy3 is linked (Upstate, U.S.A.)

D. Serum dilution solution

E. Substrate solution (TMB)

F. Washing solution: PBS-T containing 0.05% Tween

G. Standard solution: Standard solutions of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins.

Serum dilution reaction of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins of cancer patients was investigated using the above-prepared kit. The serum samples were respectively diluted appropriately using serum dilution solution (D) on the solid-phase antibody (A), added by 100 μl per well, and the concentration of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP, MSN, AFP and CRP proteins was investigated according to the sandwich type measurement as the one explained above in Example 8-4-1 using components B, C and E.

The foregoing exemplary embodiments and advantages are merely exemplary and are not to be construed as limiting the present invention. The present teaching can be readily applied to other types of apparatuses. Also, the description of the exemplary embodiments of the present inventive concept is intended to be illustrative, and not to limit the scope of the claims, and many alternatives, modifications, and variations will be apparent to those skilled in the art.

1. A kit for making a diagnosis or a prognosis of a liver cancer, comprising primers or probes binding specifically to at least one nucleotide sequence selected from the group consisting of S100, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN, or an antibody or an aptamer binding specifically to a protein encoded by the nucleotide sequence.

2. The kit of claim 1, wherein the kit is a microarray.

3. The kit of claim 1, wherein the kit is a gene amplification kit.

4. The kit of claim 1, wherein the kit is an immunoassay kit.

5. The kit of claim 4, wherein the kit is a Luminex assay kit, protein microarray kit or enzyme linked immunosorbent assay (ELISA) kit.

6. The kit of claim 1, wherein the kit further comprises primers or probes binding specifically to an AFP nucleotide sequence, or an antibody or an aptamer binding specifically to a protein encoded by the nucleotide sequence.

7. The kit of claim 6, wherein the kit further comprises primers or probes binding specifically to a CRP nucleotide sequence, or an antibody or an aptamer binding specifically to a protein encoded by the nucleotide sequence.

8. The kit of claim 1, wherein the kit further comprises primers or probes binding specifically to a CRP nucleotide sequence, or an antibody or an aptamer binding specifically to a protein encoded by the nucleotide sequence.

9. A method for diagnosing or prognosing a liver cancer, comprising a step of detecting expression of at least of nucleotide sequence selected from the group consisting of S100P, NK4, CCL20, CSPG2, PLAU, MMP12, ESM-1, ABHD7, HCAPG, CXCL-3, Col5A2, MAGEA, GSN, CDC2, CST1, MELK, ATAD2, FAP and MSN present in a biological sample of a human.

10. The method of claim 9, wherein the method is carried out in a microarray manner.

11. The method of claim 9, wherein the method is carried out in a gene amplification manner

12. The method of claim 9, wherein the method is carried out in an antigen-antibody reaction manner.

13. The method of claim 9, wherein the biological sample is blood or serum.

14. The method of claim 9, wherein the method further comprises a step of detecting expression of AFP nucleotide sequence.

15. The method of claim 14, wherein the method further comprises a step of detecting expression of CRP nucleotide sequence.

16. The method of claim 9, wherein the method further comprises a step of detecting expression of CRP nucleotide sequence.

17. A method for screening a material for preventing or treating a liver cancer, comprising:
(a) contacting a sample for analysis to a cell containing at least one nucleotide sequence selected from the group consisting of S100P, NK4, CCL20, CSPG2, PLAU, MMP12, ESM-1, ABHD7, HCCAPG, CXCL-3, Col5A2, MAGEA, GSN, CDC2, CST1, MELK, ATAD2, FAP and MSN; and
(b) measuring a level of expression of the nucleotide sequence, in which if high expression of the nucleotide sequence is inhibited, the sample is determined to be the material for preventing or treating the liver cancer.
